(19) United States
(12) Patent Application Publication
(10) Pub. No.: US 2009/0270352 A1
(43) Pub. Date: Oct. 29, 2009

(54) Tenofovir Disoproxil Hemi-Fumaric Acid Co-Crystal

(75) Inventors: Evanthia Dova, Amsterdam (NL); Jaroslaw Marek Mazurek, Rijswijk (NL); Johnny Anker, Anna Paulowna (NL)

Correspondence Address: HOFFMANN & BARON, LLP, 6900 JERICHO TURNPIKE, SYOSSET, NY, 11791, US

(73) Assignee: ULTIMORPHIX TECHNOLOGIES B.V., Amsterdam (NL)

(21) Appl. No.: 12/301,872

(22) PCT Filed: May 21, 2008
(86) PCT No.: PCT/NL08/00132

§ 371 Date: Feb. 24, 2009

Related U.S. Application Data
(60) Provisional application No. 60/939,544, filed on May 22, 2007.
(60) Provisional application No. 60/945,612, filed on Jun. 22, 2007.
(60) Provisional application No. 60/947,502, filed on Jul. 2, 2007.
(60) Provisional application No. 60/951,316, filed on Jul. 23, 2007.

(51) Int. Cl. A61K 31/675; A61P 31/18; C07D 473/00

(52) U.S. Cl. 514/81; 544/277

(57) ABSTRACT

The present invention provides a novel crystalline form of Tenofovir disoproxil fumarate (Tenofovir DF), designated Co-crystal TDFA 2:1, methods for the preparation thereof and its use in pharmaceutical applications, in particular in anti-HIV medicaments. The crystalline form TDFA 2:1 can be used in combination with other anti-HIV medicaments such as Efavirenz, Emtricitabine, Ritonavir and/or TMC114.

### FIELD OF THE INVENTION

The present invention relates to a novel co-crystalline composition of tenofovir disoproxil and fumaric acid in a 2:1 molar ratio, methods for its preparation and its formulation and application in the field of medicine, in particular antiviral medicines.

### BACKGROUND OF THE INVENTION

Tenofovir disoproxil fumarate (DF) is a nucleotide reverse transcriptase inhibitor approved in the United States for the treatment of HIV-I infection alone or in combination with other antiretroviral agents. Tenofovir disoproxil DF is sold under the VIREAD® trade name (Gilead Science, Inc.) and present in combination with other anti-viral agents in the TRUVADA® and ATRIPLA™ anti-HIV drugs.

Among the anti-HIV drugs which have been developed are those which target the HIV reverse transcriptase (RT) enzyme or protease enzyme, both of which enzymes are necessary for the replication of the virus. Examples of RT inhibitors include nucleoside/nucleotide RT inhibitors (NRTIs) and non-nucleoside RT inhibitors (NNRTIs). Currently, HIV-infected patients are routinely being treated with three-drug combinations. Regimens containing (at least) three NRTIs; two NRTIs in combination with one or two protease inhibitors (PI)(s); or two NRTIs in combination with a NNRTI, are widely used. When two or more PIs are used in these combinations, one of the PIs is often ritonavir, given at a low sub-therapeutic dose, which acts as an effective inhibitor of the elimination of the other PI(s) in the regimen, resulting in maximal suppression of the virus and thereby reducing the emergence of resistance.

Clinical studies have shown that three-drug combinations of these anti-HIV drugs are much more effective than one drug used alone or two-drug combinations in preventing disease progression and death. Numerous studies of drug combinations with various combinations of such drugs have established that such combinations greatly reduce disease progression and deaths in people with HIV infections. The name now commonly given to combinations of anti-HIV drugs is HAART (Highly Active Anti-Retroviral Therapy).

Tenofovir disoproxil fumarate, also known as Tenofovir DF, Tenofovir disoproxil, TDF, Bis-POC-PMPA (U.S. Pat. Nos. 5,935,946, 5,922,695, 5,977,089, 6,043,230, 6,069,249) is a prodrug salt of tenofovir. The chemical name of tenofovir disoproxil fumarate is 9-[(R)-2-[[bis[[(isopropoxycarbonyl)oxy]methoxy]phosphinyl]-methoxy]propyl] adenine fumarate (1:1). The CAS Registry number is 202138-50-9. It has a molecular formula of C₁₉H₃₀N₅O₁₀P.C₄H₄O₄ and a molecular weight of 635.52. It has the following structural formula:

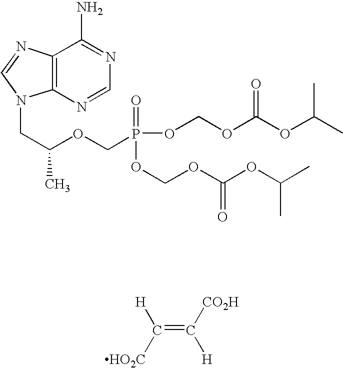

A crystalline form of Tenofovir DF is described inter alia in WO99/05150, EP998480, and U.S. Pat. No. 5,935,946. This crystalline form (Gilead 1) is characterised as having XRPD peaks at about 4.9, 10.2, 10.5, 18.2, 20.0, 21.9, 24.0, 25.0, 25.5, 27.8, 30.1 and 30.4. Furthermore these crystals are described as opaque or off-white and exhibit a DSC absorption peak at about 118° C. with an onset at about 116° C. and an IR spectrum showing characteristic bands expressed in reciprocal centimetres at approximately 3224, 3107-3052, 2986-2939, 1759, 1678, 1620, 1269 and 1102. Bulk densities have been described of about 0.15-0.30 g/mL, usually about 0.2-0.25 g/mL.

After analysis of several commercially available products containing tenofovir DF, it was found that these contained mixtures of solid forms of tenofovir DF in varying ratios. Indications have been found by the present inventors that the solid form of Tenofovir DF in commercially available products is generally a mixture of at least two forms. It has also been found that one of these forms experiences a conversion of its crystalline form into the other form when put under stress, such as increased temperature and/or humidity. It is believed by the present inventors that the presence of water will induce or enhance the conversion of one form into the other. This suggests that the solid form currently used in the marketed product is not stable or at least has a reduced stability. The bulk molar ratio of tenofovir disoproxil to fumaric acid in the commercially available products is generally indicated as 1:1.

### SUMMARY OF THE INVENTION

The present invention relates to a novel co-crystal of tenofovir disoproxil and fumaric acid in a 2:1 molar ratio, (TDFA 2:1). The invention differs from tenofovir DF, which is a 1:1 fumarate salt. The TDFA 2:1 co-crystal of the invention is more stable and is less hygroscopic than the presently known crystalline form of tenofovir DF (Gilead 1).

### DESCRIPTION OF THE DRAWINGS

FIG. 1A illustrates the X-Ray Powder Diffraction pattern of the co-crystal of tenofovir disoproxil and fumaric acid in a 2:1 molar ratio, (TDFA 2:1).

FIG. 1B illustrates the DSC thermogram of the co-crystal of tenofovir disoproxil and fumaric acid in a 2:1 molar ratio, (TDFA 2:1).

FIG. 1C illustrates the TGA thermogram of the co-crystal of tenofovir disoproxil and fumaric acid in a 2:1 molar ratio, (TDFA 2:1).

FIG. 1D illustrates the molecular structure of free base-free acid-free base entity from the co-crystal of tenofovir disoproxil and fumaric acid in a 2:1 molar ratio, (TDFA 2:1), as determined from single crystal data.

FIG. 1E illustrates the crystal packing for the co-crystal of tenofovir disoproxil and fumaric acid in a 2:1 molar ratio, (TDFA 2:1).

FIG. 1F illustrates the Raman spectrum for the co-crystal of tenofovir disoproxil and fumaric acid in a 2:1 molar ratio, (TDFA 2:1).

FIG. 2A illustrates the X-ray powder diffraction pattern obtained from a ground tablet of Viread.

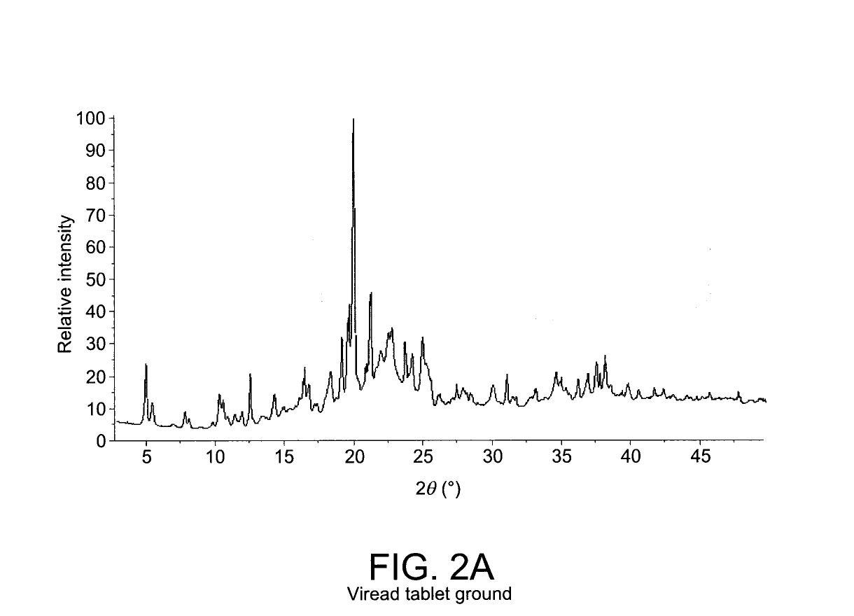

FIG. 2B illustrates the X-ray powder diffraction pattern obtained from a tablet of Viread after removal of the coating.

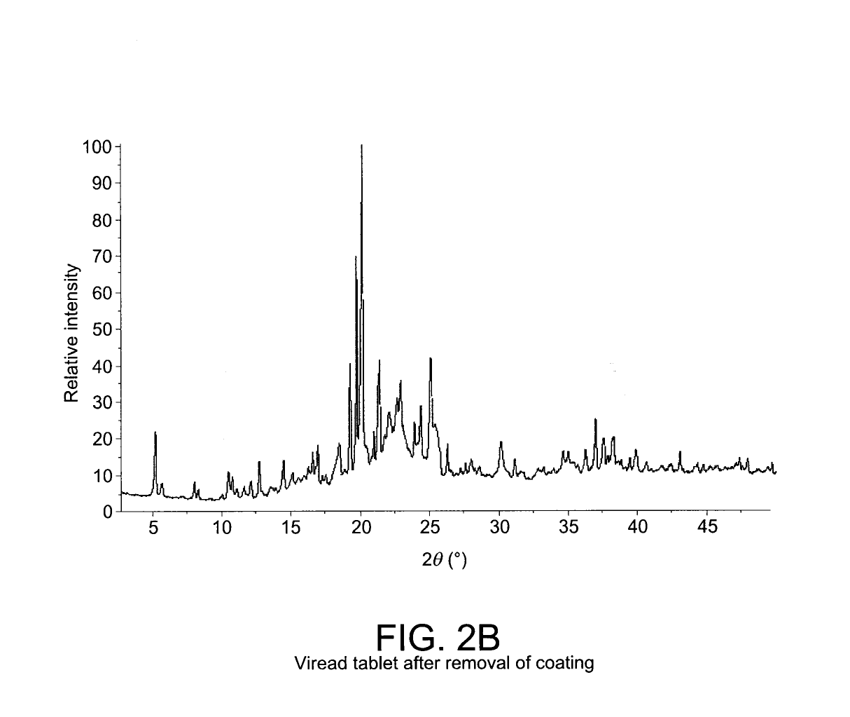

FIG. 2C illustrates the X-ray powder diffraction pattern obtained from a ground tablet of Truvada.

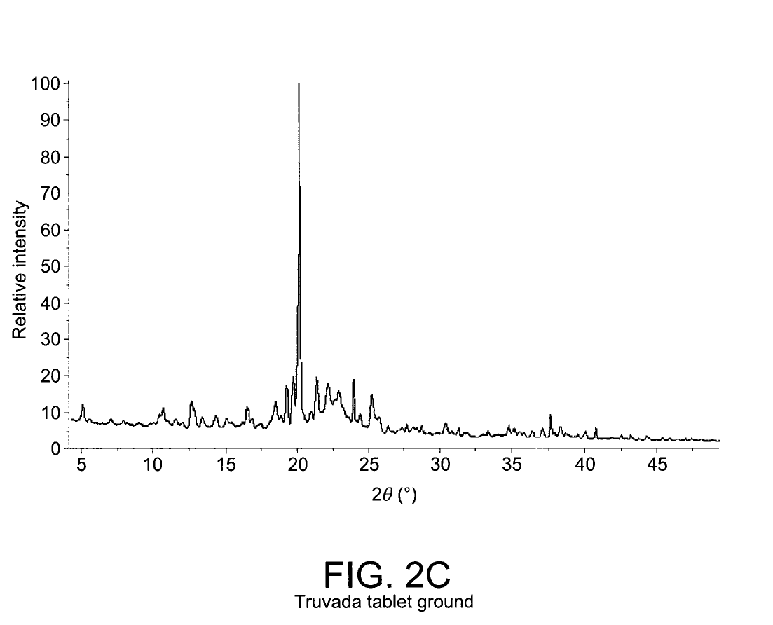

FIG. 3 DVS plot of the sorption (diamond) and desorption (square) behaviour of form TDFA 2:1

FIG. 4 Experimental XRPD patterns of form TDFA 2:1 before (top) and after (bottom) DVS measurement.

FIG. 5 Pharmacokinetic data, indicating bioequivalence of TFDA 2:1.

### DETAILED DESCRIPTION OF THE INVENTION

### The Tenofovir disoproxil/Fumaric Acid Co-Crystal (TDFA 2:1)

The invention relates to a co-crystal of tenofovir disoproxil with fumarate wherein two units of tenofovir disoproxil are co-crystallised with one unit of fumaric acid with an empirical formula of 2 C₁₉H₃₀N₅O₁₀P.C₄H₄O₄. This co-crystal is a hemifumaric acid co-crystal of tenofovir disoproxil. In one aspect, the present invention provides a substantially pure composition, particularly a co-crystal, of tenofovir disoproxil and fumaric acid in a 2:1 molar ratio, (TDFA 2:1). A co-crystal is a crystalline entity in which more than one molecular substance is incorporated into the unit cell. This normally excludes: salts such as tenofovir DF, which are distinguished by proton transfer, giving electrostatic linkage between oppositely-charged ions, and solvates, which are associations of substrates with solvents from which they are crystallized although the bonding mechanisms can be similar to those in co-crystals. See, e.g. Visheweshwar, P.; McMahon, J. A.; Bis, J. A.; Zaworotko, M. J. (2006) J. Pharm. Sci. 95(3), 499-516.

As discussed above, the novel solid form TDFA 2:1 of the present invention is, independently, in a substantially pure form, preferably substantially free from other amorphous, and/or crystalline solid forms such as the solid forms as described in the prior art as referred herein before, i.e. Gilead 1 or ULT-1, as described herein elsewhere. In this respect, “substantially pure” relates to at least about 80%, 85%, 90%, 95%, 96%, 97%, 98%, or 99% of the pure compound. In this respect, “substantially free from other amorphous, and/or crystalline solid forms” means that no more than about 20%, 15%, 10%, 5%, 4%, 3%, 2%, 1% of these other amorphous, and/or crystalline solid forms are present in the form according to the invention.

The co-crystal of the present invention is a co-crystal at temperatures below room temperature, preferably at temperatures around 120K. The co-crystal of the present invention is also a co-crystal at more elevated temperatures, for instance room temperature. Experimental XRPD pattern and single crystal structure at room temperature show that there is no structural phase transition between 120K and room temperature, and the differences in the XRPD patterns at these temperatures are due to thermal expansion. On basis of the above it is concluded that TDFA 2:1 is a co-crystal at room temperature (between 15 and 40 degrees Celsius, depending on the geographical location of the measurement).

TDFA 2:1 is characterised by the selection of at least one, preferably at least two, more preferably at least three, even more preferably at least four, particularly preferred at least five and most preferred six X-ray powder diffraction peaks selected from the group consisting of 7.9, 9.8, 11.0, 12.0, 13.7, 14.3, 16.1, 16.8, 18.0, 19.2, 20.4, 21.2, 21.7, 22.6, 23.4, 24.3, 25.4, 27.6, degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine, particularly preferred at least ten and most preferred eleven X-ray powder diffraction peaks are selected from the above group. In a more preferred embodiment, at least twelve, more preferably at least thirteen, even more preferably at least fourteen, particularly preferred at least fifteen and most preferred sixteen, seventeen or eighteen X-ray powder diffraction peaks are selected from the above group.

Preferably, TDFA is characterised by the selection of at least one, preferably at least two, more preferably at least three, even more preferably at least four, particularly preferred at least five and most preferred six X-ray powder diffraction peaks selected from the group consisting of 7.82, 8.09, 11.95, 16.80, 21.20, 22.52, 24.29°2θ. The 2θ positions are calculated from the single crystal structure of TDFA 2:1 at room temperature using a wavelength of 1.54178 Å. In an experimental XRPD pattern, there may be deviations from the above listed values due to experimental settings and peak overlap.

TDFA 2:1 can be characterised by the following set of X-ray diffraction peaks and, optionally, by the associated intensities:

TABLE 1

| | | | Preferred embodiment | |
|---|---|---|---|---|
| Peak ID | Angle (2θ) | Intensity* | Angle (2θ) | Intensity* |
| 1 | 7.9 | H | 7.86 | H |
| 2 | 9.8 | M | 9.82 | M |
| 3 | 11.0 | L | 10.98 | L |
| 4 | 12.0 | H | 11.96 | H |
| 5 | 13.7 | L | 13.70 | L |
| 6 | 14.3 | H | 14.28 | H |
| 7 | 16.1 | M | 16.10 | M |
| 8 | 16.8 | H | 16.76 | H |
| 9 | 18.0 | M | 18.02 | M |
| 10 | 19.2 | M | 19.18 | M |
| 11 | 20.4 | M | 20.44 | M |
| 12 | 21.2 | H | 21.18 | H |
| 13 | 21.7 | M | 21.66 | M |
| 14 | 22.6 | H | 22.60 | H |
| 15 | 23.4 | L | 23.42 | L |
| 16 | 24.3 | H | 24.30 | H |
| 17 | 25.4 | H | 25.36 | H |
| 18 | 27.6 | L | 27.60 | L |

| | | | |
|---|---|---|---|
| normalised intensity | L | 0 | 30 |
| values: | M | 30 | 60 |
| | H | 60 | 100 |

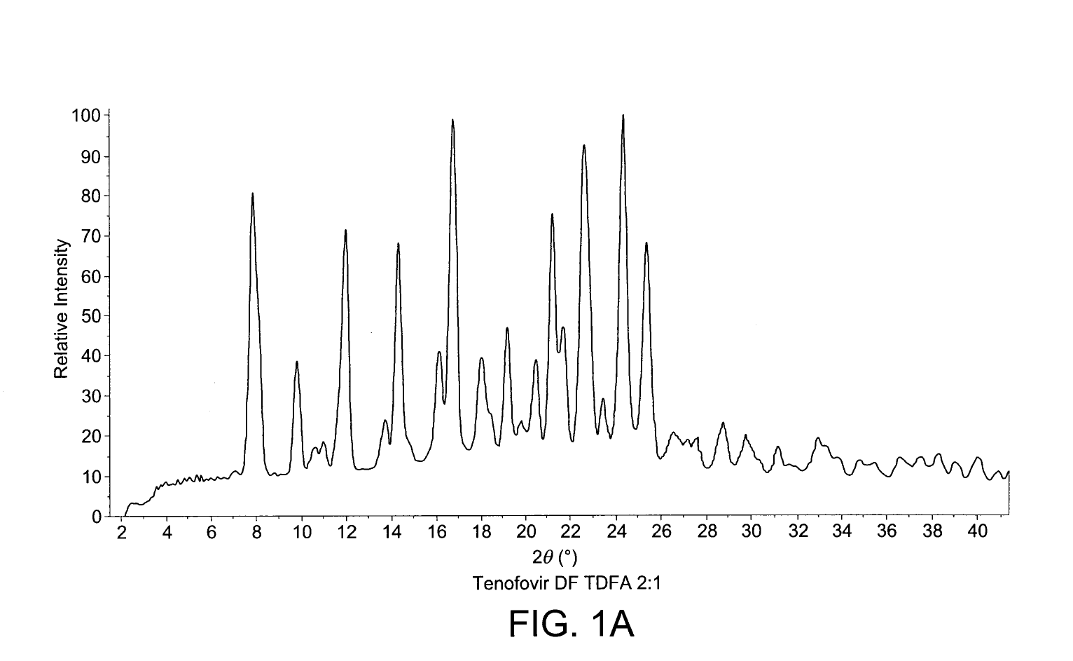

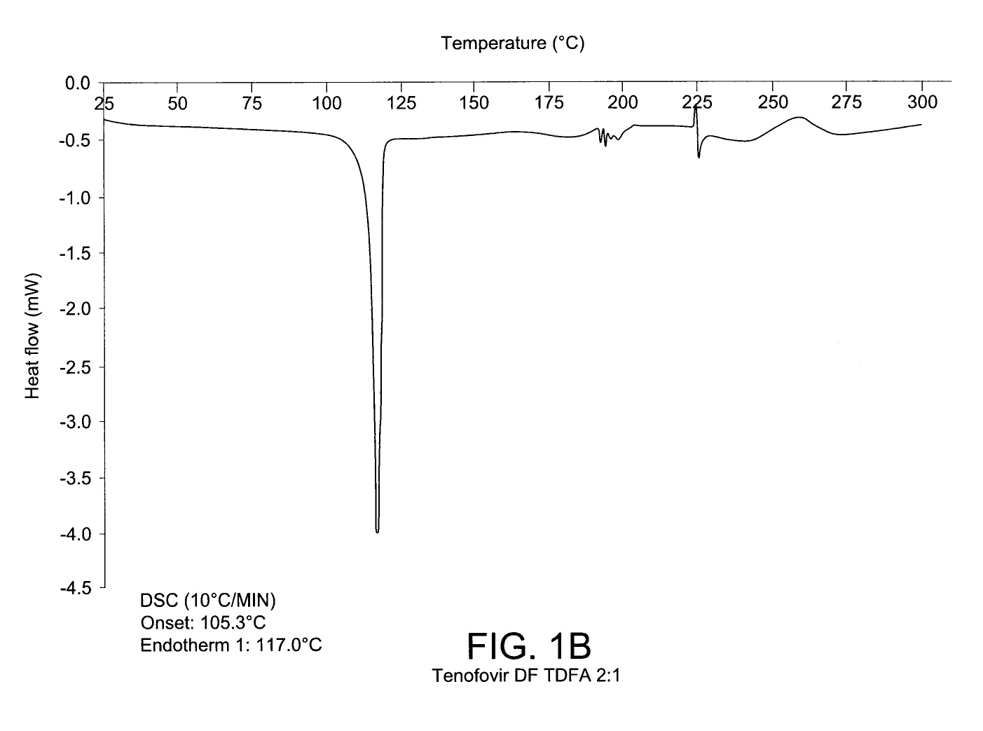

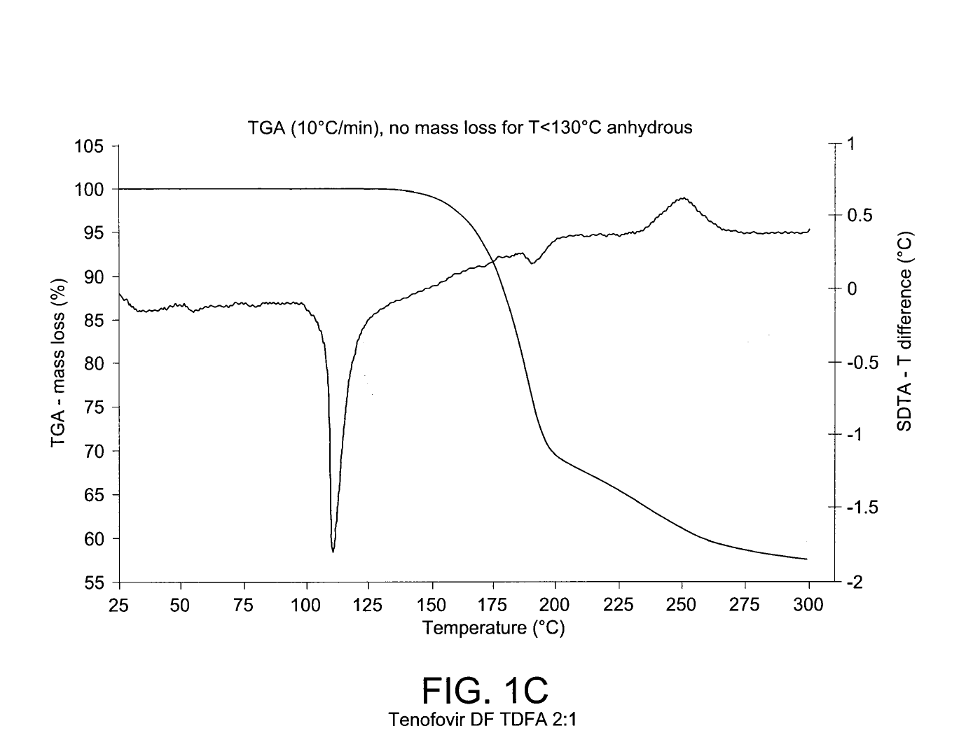

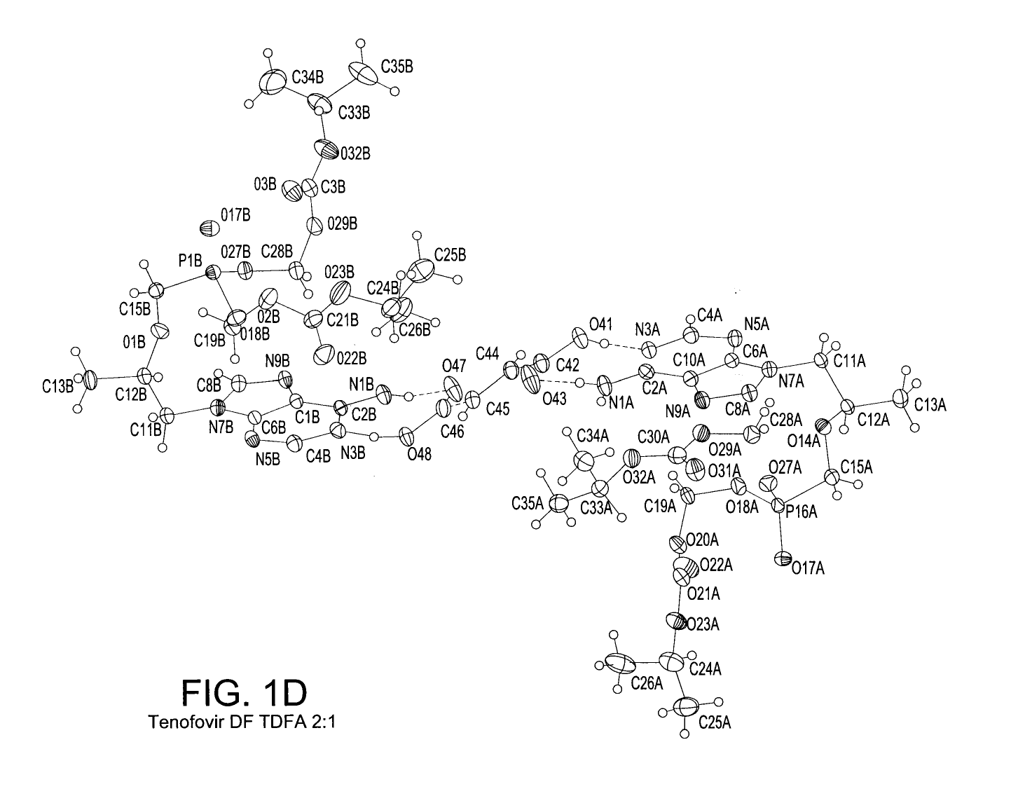

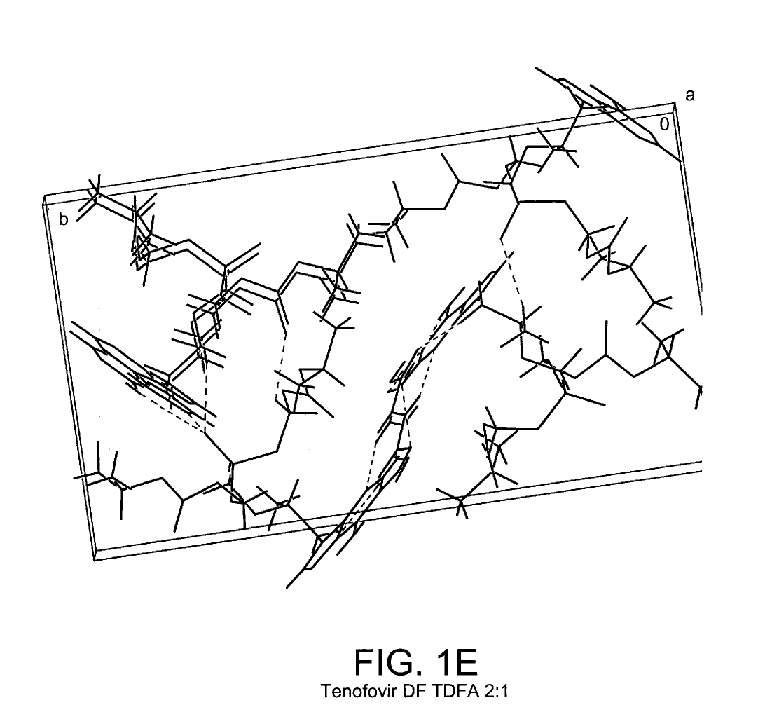

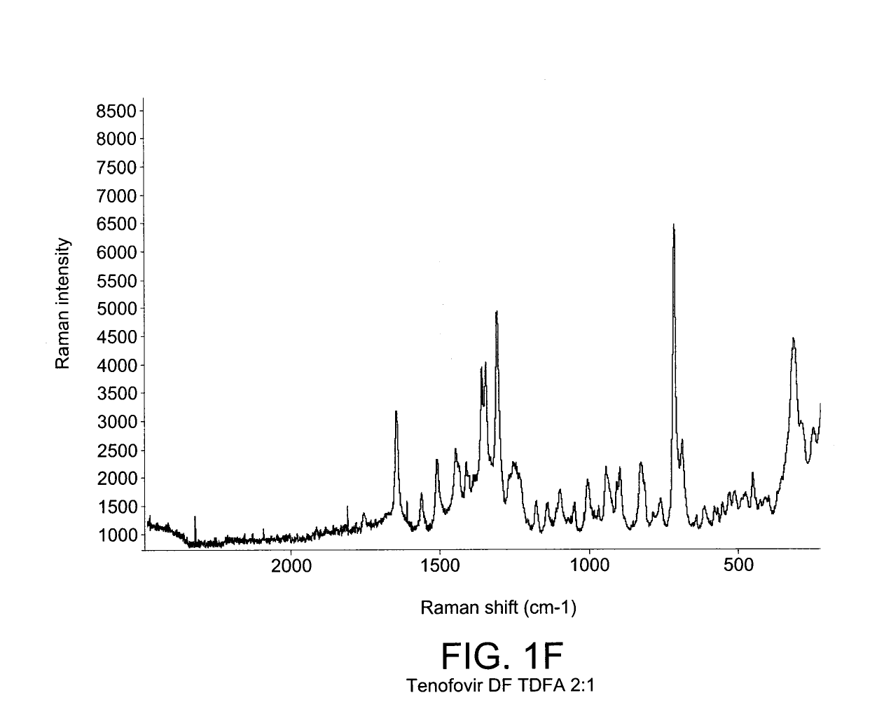

In another embodiment, TDFA 2:1 can be characterised by an X-ray diffraction pattern substantially according to FIG. 1A.

In another embodiment, TDFA 2:1 can be characterised by an DSC substantially according to FIG. 1B.

In another embodiment, Form TDFA 2:1 can be characterised by an TGA substantially according to FIG. 1C.

In another embodiment, Form TDFA 2:1 of the present invention can be characterised by DSC with an onset at 105.3° C. and a characterising peak at 117.0° C. From the thermal analysis, it is concluded that the co-crystal TDFA 2:1 is unsolvated.

The present invention in one aspect relates to a method for the preparation of the co-crystal TDFA 2:1 comprising the steps of dissolving or mixing tenofovir DF in a suitable solvent or mixture thereof as in Table I and crystallising tenofovir DF Form TDFA 2:1 by evaporation of the solvent.

The present invention in another aspect relates to a method for the preparation of the co-crystal TDFA 2:1 comprising the steps of dissolving or mixing tenofovir DF in a suitable solvent or mixture thereof as in Table II and crystallising TDFA 2:1 by cooling and/or evaporation crystallization of a saturated solution.

The present invention in one aspect relates to a method for the preparation of the co-crystal TDFA 2:1 of tenofovir DF comprising the steps of dissolving or mixing tenofovir DF in a suitable solvent or mixture thereof as in Table III and crystallising TDFA 2:1 by anti-solvent addition as in Table III.

The present invention in another aspect relates to a method for the preparation of the co-crystal TDFA 2:1 comprising the steps of dissolving or mixing tenofovir DF in a suitable solvent or mixture thereof as outlined herein elsewhere (paragraph on solvents) crystallising TDFA 2:1 by slurry crystallisation and/or seed crystallisation.

The co-crystal of the invention has also been characterized in one aspect relates to the single-crystal structure of TDFA 2:1 as depicted in FIGS. 1D and/or 1E and/or in the table 2 and 3:

TABLE 2
Crystal data and structure refinement for TDFA 2:1.

| Empirical formula | 2C₁₉H₃₀N₅O₁₀P•C₄H₄O₄ |
|---|---|
| Formula weight | 1154.97 |
| Temperature (K) | 120(2) |
| Wavelength (Å) | 0.71073 |
| Crystal system | Monoclinic |
| Space group | P 2₁ |
| Unit cell dimensions (Å) | 9.7710(2) [9.8490(2)]* |
| | 22.1490(2) [22.6250(6)] |
| | 12.4680(2) [12.5350(4)] |
| | 95.1490(3) [95.122(1)] |
| Volume (Å³) | 2687.41(4) [2782.1(2)] |
| Z | 2 |
| Density (calculated) | 1.427 [1.379] |
| F(000) | 1216 |
| Crystal size (mm) | 0.3 × 0.3 × 0.22 |
| Theta range for data | 2.5 → 35 |
| collection (°) | |
| Reflections collected | 40343 |
| Independent reflections | 23056 [R(int) = 0.0226] |
| Data/restraints/parameters | 23056/1/959 |
| Goodness-of-fit on F² | 1.036 |
| Final R indices [I > 2sigma(I)] | R1 = 0.0352, wR2 = 0.0811 |
| R indices (all data) | R1 = 0.0394, wR2 = 0.0838 |
| Absolute structure parameter | −0.02(3) |

*in square brackets the unit cell dimensions at room temperature

In one aspect the invention relates further to TDFA 2:1 substantially pure and preferably free from Tenofovir DF form ULT-1 (as described in applicant's co-pending application U.S. 60/873,267 incorporated herein by reference). Tenofovir DF form ULT-1 as disclosed in U.S. 60/873,267 can be characterised by the selection of at least one, preferably at least two, more preferably at least three, even more preferably at least four, particularly preferred at least five and most preferred six X-ray powder diffraction peaks selected from the group consisting of 5.0, 5.5, 10.3, 10.6, 10.9, 11.4, 14.2, 17.3, 18.3, 19.9, 22.0, 22.9, 25.0, 27.9, 30.1 degrees two-theta+/−0.3 degrees two-theta, preferably +/−0.2 degrees two-theta, more preferably +/−0.1 degrees two-theta, most preferably +/−0.05 degrees two-theta. In a preferred embodiment, at least seven, more preferably at least eight, even more preferably at least nine, particularly preferred at least ten and most preferred eleven X-ray powder diffraction peaks are selected from the above group. In a more preferred embodiment, at least twelve, more preferably at least thirteen, even more preferably at least fourteen, particularly preferred at least fifteen X-ray powder diffraction peaks are selected from the above group.
In a preferred embodiment of the present invention, TDFA 2:1 is substantially free from a solid form tenofovir DF form ULT-1. In a preferred embodiment of the present invention, TDFA 2:1 is substantially free from a solid form characterised by having an X-ray peak at 5.0 and/or 5.5 degrees two-theta+/−0.1 degrees two-theta. In a further preferred embodiment, TDFA 2:1 is substantially free from a solid form characterised by having an X-ray peak at 4.9 and/or 5.4 degrees two-theta+/−0.1 degrees two-theta. In a further preferred embodiment, TDFA 2:1 is substantially free from a solid form characterised by having an X-ray peak at 4.97 and/or 5.44 degrees two-theta+/−0.1 degrees two-theta.

In one aspect the invention relates to a pharmaceutical composition comprising form TDFA 2:1 substantially pure, preferably obtained from Tenofovir DF form ULT-1 (as described herein elsewhere and in applicant's co-pending application U.S. 60/873,267).

In one aspect the invention relates to a process for the preparation of form TDFA 2:1 from the starting material Tenofovir DF obtained from Cipla by recrystallisation to form a 2:1 hemifumaric acid co-crystal from organic solvents as listed in one or more of the tables I, II, and/or III or mixtures thereof.

In one aspect the invention relates to a method for the preparation of from TDFA 2:1 from Tenofovir DF form ULT-1 by crystallisation in an aqueous environment.

The single crystal of the co-crystal was obtained by slow evaporation of saturated solution of tenofovir DF in water, methanol, isopropyl acetate, (R)-(−)-2-octanol at room temperature or lower temperature, preferably at 5° C. In another embodiment the saturated solution is cooled with a cooling rate of 1° C./h to 5° C. and then aged at this temperature for several days. It is also possible to obtain the co-crystal TDFA 2:1 from the solvents listed in Tables I, II and III.

Solvents

In certain embodiments of the method for the preparation of TDFA 2:1 of the present invention, the solvents for evaporation crystallisation, hot filtration anti-solvent addition, seed crystallisation and/or slurry crystallisation are preferably selected from the group consisting of: (R)-(−)-2-octanol, 1,2-diethoxyethane, 1,2-dimethoxyethane, 1,4-dioxane, 1-butanol, 1-heptanol, 1-hexanol, 1-methoxy-2-propanol, 1-nitropropane, 1-octanol, 2,2,2-trifluoroethanol, 2-butanone, 2-ethoxyethanol, 2-ethoxyethyl acetate, 2-hexanol, 2-methoxyethanol, 2-Nitropropane, 2-pentanol, 2-propanol, 4-hydroxy-4-methyl-2-pentanon, acetone, acetonitrile, butyronitrile, cyclohexanol, cyclopentanol, cyclopentanone, diethylene glycol dimethylether, dimethylcarbonate, dimethylcarbonate, ethanol, ethyl formate, ethylacetate, ethylene glycol monobutyl ether, dichloromethane, furfuryl alcohol, isobutanol, isopropyl acetate, methanol, methoxyethyl acetate, methyl acetate, methyl butyrate, methyl propionate, 2-methyl-4-pentanol, N,N-dimethylacetamide, N,N-dimethylformamide, nitrobenzene, nitroethane, nitromethane, N-methylpyrrolidone, propionitrile, propyl acetate, propylene glycol methyl ether acetate, tert-butanol, tetrahydrofuran, tetrahydrofurfurylalcohol, tetrahydropyran, Water and mixtures thereof.

In certain embodiments of the method for the preparation of TDFA 2:1 of the present invention, the solvents for evaporation crystallisation, hot filtration anti-solvent addition, seed crystallisation and/or slurry crystallisation are more preferably selected from the group consisting of:

(R)-(−)-2-octanol, 1,2-diethoxyethane, 1,2-dimethoxyethane, 1,4-dioxane, 1-butanol, 1-nitropropane, 1-propanol, 2-butanone, 2-ethoxyethyl acetate, 2-methyl-4-pentanol, 2-nitropropane, 2-propanol, acetone, acetonitrile, cyclopentanol, ethanol, isobutanol, isopropyl acetate, methanol, methoxy-2-1-propanol, methyl propionate, N,N-dimethylacetamide, N,N-dimethylformamide, nitromethane, tert-butanol, tetrahydrofuran, water, 1,2-dichloroethane, 2,6-dimethyl-4-heptanone, Amyl ether, Butyl benzene, Chloroform, Dichloromethane, hexafluorobenzene, n-heptane, N-methylpyrrolidone, tert-butyl methyl ether, toluene, cyclopentanone.

In certain embodiments of the method for the preparation of TDFA 2:1 of the present invention, the solvents for hot filtration crystallisation are preferably selected from the group consisting of: (R)-(−)-2-octanol, 1,2-diethoxyethane, 1,2-dimethoxyethane, 1,4-dioxane, 1-Butanol, 1-nitropropane, 1-propanol, 2-butanone, 2-ethoxyethyl acetate, 2-methyl-4-pentanol, 2-nitropropane, 2-propanol, acetone, acetonitrile, cyclopentanol, ethanol, isobutanol, isopropyl acetate, methanol, methoxy-2-1-Propanol, methyl propionate, N,N-dimethylacetamide, N,N-dimethylformamide, nitromethane, tert-butanol, tetrahydrofuran, water and mixtures thereof.

In certain embodiments of the method for the preparation of TDFA 2:1 of the present invention, the solvents for solvent/anti-solvent crystallisation are preferably selected from the group consisting of: 1,2-dichloroethane, 1,2-dimethoxyethane, 1,4-dioxane 2,6-dimethyl-4-heptanone, 2-butanone, acetone, acetonitrile, amyl ether, butyl benzene, chloroform, cyclohexane, cyclohexane, dichloromethane, hexafluorobenzene, methanol, n-heptane, nitromethane, N-methylpyrrolidone, tert-butyl methyl ether, tetrahydrofuran, toluene, water and mixtures thereof.

In certain embodiments of the method for the preparation of TDFA 2:1 of the present invention, the anti-solvents for anti-solvent crystallisation are preferably selected from the group consisting of: 1,2-dichloroethane, 2,6-dimethyl-4-heptanone, acetone, amyl ether, butyl benzene, chloroform, cyclohexane, dichloromethane, hexafluorobenzene, n-heptane, nitromethane, tert-butyl methyl ether, toluene and mixtures thereof.

In certain embodiments of the method for the preparation of TDFA 2:1 of the present invention, the solvents for seeding crystallisation are preferably selected from the group consisting of: methanol, water, 1,4-dioxane, acetonitrile, 2-ethoxyethylacetate, 2-methyl-4-pentanol, tetrahydrofuran, butyl benzene, amylether, tert-butyl methyl ether, cyclopentanone and mixtures thereof.

In certain embodiments of the method for the preparation of TDFA 2:1 of the present invention, the solvents for slurrying crystallisation are preferably selected from the group consisting of: water, methanol, acetonitrile, 1,4-dioxane and mixtures thereof.

Pharmaceutical Formulations.

The present invention further relates to pharmaceutical formulations comprising the novel crystalline forms of tenofovir DF.

Pharmaceutical formulations of the present invention contain TDFA 2:1 as disclosed herein. The invention also provides pharmaceutical compositions comprising one or more of the crystal forms according to the present invention. Pharmaceutical formulations of the present invention contains one or more of the crystal form according to the present invention as active ingredient, optionally in a mixture with other crystal form(s).

The pharmaceutical formulations according to the invention, may further comprise, in addition to the form TDFA 2:1 additional pharmaceutical active ingredients, preferably Anti-HIV agents and more preferably Efavirenz, Emtricitabine, Ritonavir and/or TMC114.

In addition to the active ingredient(s), the pharmaceutical formulations of the present invention may contain one or more excipients. Excipients are added to the formulation for a variety of purposes.

Diluents increase the bulk of a solid pharmaceutical composition, and may make a pharmaceutical dosage form containing the composition easier for the patient and caregiver to handle. Diluents for solid compositions include, for example, microcrystalline cellulose (e.g. Avicel(R)), micro fine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit(R)), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. Carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel(R)), hydroxypropyl methyl cellulose (e.g. Methocel(R)), liquid glucose, magnesium aluminum silicate, maltodextrin, methylcellulose, polymethacrylates, povidone (e.g. Kollidon(R), Plasdone(R)), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol(R), Primellose(R)), colloidal silicon dioxide, croscarmellose sodium, crospovidone (e.g. Kollidon(R), Polyplasdone(R)), guar gum, magnesium aluminum silicate, methyl cellulose, microcrystalline cellulose, polacrilin potassium, powdered cellulose, pregelatinized starch, sodium alginate, sodium starch glycolate (e.g. Explotab(R)) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate. Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid. Solid and liquid compositions may also be dyed using any pharmaceutically acceptable colorant to improve their appearance and/or facilitate patient identification of the product and unit dosage level.

In liquid pharmaceutical compositions of the present invention, the crystalline forms according to the present invention and any other solid excipients are suspended in a liquid carrier such as water, vegetable oil, alcohol, polyethylene glycol, propylene glycol, glycerin or mixtures thereof, as long as the presently described crystalline from is maintained therein, i.e. does not dissolve.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methylcellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste. Preservatives and chelating agents such as alcohol, sodium benzoate, butylated hydroxyl toluene, butylated hydroxyanisole and ethylenediamine tetraacetic acid may be added at levels safe for ingestion to improve storage stability. According to the present invention, a liquid composition may also contain a buffer such as gluconic acid, lactic acid, citric acid or acetic acid, sodium gluconate, sodium lactate, sodium citrate or sodium acetate. Selection of excipients and the amounts used may be readily determined by the formulation scientist based upon experience and consideration of standard procedures and reference works in the field.

For infections of the eye or other external tissues, e.g. mouth and skin, the formulations are preferably applied as a topical ointment or cream containing the active ingredient(s) in an amount of, for example, 0.01 to 10% w/w (including active ingredient(s) in a range between 0.1% and 5% in increments of 0.1% w/w such as 0.6% w/w, 0.7% w/w, etc), preferably 0.2 to 3% w/w and most preferably 0.5 to 2% w/w. When formulated in an ointment, the active ingredients may be employed with either a paraffinic or a water-miscible ointment base.

Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the emulsifying wax, and the wax together with the oil and fat make up the emulsifying ointment base which forms the oily dispersed phase of the cream formulations.

Emulgents and emulsion stabilisers suitable for use in the formulation of the present invention include Tween8 60, Spans 80, cetostearyl alcohol, benzyl alcohol, myristyl alcohol, glyceryl monostearate and sodium lauryl sulfate.

The choice of suitable oils or fats for the formulation is based on achieving the desired cosmetic properties. Thus the cream should preferably be a non-greasy, non-staining and washable product with suitable consistency to avoid leakage from tubes or other containers.

Straight or branched chain, mono- or dibasic alkyl esters such as diisoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the active ingredient. The active ingredient is suitably present in such formulations in a concentration of 0.01 to 20%, in some embodiments 0.1 to 10%, and in others about 1.0% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for nasal or inhalational administration wherein the carrier is a solid include a powder having a particle size for example in the range 1 to 500 microns (including particle sizes in a range between 20 and 500 microns in increments of 5 microns such as 30 microns, 35 microns, etc). Suitable formulations wherein the carrier is a liquid, for administration as for example a nasal spray or as nasal drops, include aqueous or oily solutions of the active ingredient.

Formulations suitable for aerosol administration may be prepared according to conventional methods and may be delivered with other therapeutic agents. Inhalational therapy is readily administered by metered dose inhalers.

Formulations suitable for vaginal administration may be presented as pessaries, tampons, creams, gels, pastes, foams or spray formulations containing in addition to the active ingredient such carriers as are known in the art to be appropriate.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art. A composition for tabletting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tabletted/compressed, or other excipients may be added prior to tabletting, such as a glidant and/or a lubricant.

A tabletting composition may be prepared conventionally by dry blending. For example, the blended composition of the actives and excipients maybe compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

Moreover, the crystalline form according to the present invention can be formulated for administration to a mammal, preferably a human, via injection. The crystalline form according to the present invention may be formulated, for example, as a viscous liquid solution or suspension, for injection. The formulation may contain solvents. Among considerations for such solvent include the solvent's physical and chemical stability at various pH levels, viscosity (which would allow for syringeability), fluidity, boiling point, miscibility and purity. Suitable solvents include alcohol USP, benzyl alcohol NF, benzyl benzoate USP and Castor oil USP. Additional substances may be added to the formulation such as buffers, solubilizers, antioxidants, among others. Ansel et al., Pharmaceutical Dosage Forms and Drug Delivery Systems, 7th Ed.

The present invention also provides pharmaceutical formulations comprising the crystalline form according to the present invention, optionally in combination with other polymorphic forms or co-crystals, to be used in a method of treatment of a mammal, preferably a human, in need thereof. A pharmaceutical composition of the present invention comprises the crystalline form TDFA 2:1. The crystalline form according to the present invention may be used in a method of treatment of a mammal comprising administering to a mammal suffering from the ailments described herein before a therapeutically effective amount of such pharmaceutical composition. The invention further relates to the use of the crystalline form of the invention for the preparation of a medicament for the treatment of the ailments described herein before, in particular HIV.

Having described the invention with reference to certain preferred embodiments, other embodiments will become apparent to one skilled in the art from consideration of the specification. The invention is further defined by reference to the following examples describing in detail the preparation of the compounds of the present invention. It will be apparent to those skilled in the art that many modifications, both to materials and methods, may be practiced without departing from the scope of the invention.

### EXAMPLES

### Experimental Conditions

### X-Ray Powder Diffraction:

XRPD patterns were obtained using a T2 high-throughput XRPD set-up by Avantium technologies, The Netherlands. The plates were mounted on a Bruker GADDS diffractometer equipped with a Hi-Star area detector. The XRPD platform was calibrated using Silver Behenate for the long d-spacings and Corundum for the short d-spacings.

Data collection was carried out at room temperature using monochromatic CuK(alpha)radiation (1.54178 Å) in the two-theta region between 1.5° and 41.5°. The diffraction pattern of each well is collected in two two-theta ranges (1.5°≦2θ≦21.5° for the first frame, and 19.5°≦2θ≦41.5° for the second) with an exposure time of 120 s for each frame. One of ordinary skill in the art understands that experimental differences may arise due to differences in instrumentation, sample preparation, or other factors. Typically XRPD data are collected with a variance of about 0.3 degrees two-theta, preferable about 0.2 degrees, more preferably 0.1 degrees, even more preferable 0.05 degrees. This has consequences for when X-ray peaks are considered overlapping.

### High-Resolution X-Ray Powder Diffraction:

The High resolution powder patterns were collected on the D8 Advance system in the Brag-Brentano geometry equipped with LynxEye solid state detector. The radiation used for collecting the data was CuK(alpha1=1.54056 A) monochromatized by the Germanium crystal. The patterns were collected in various 2θ ranges, starting from about 2-4°2θ until about 60-65°2θ, with a step in the range of 0.04-0.16°2θ without further processing. All patterns were taken at Room Temperature, approximately 295K.

### Single-Crystal X-Ray Diffraction

Suitable single crystals were selected and glued to a glass fibre, which was then mounted on an X-ray diffraction goniometer. X-ray diffraction data were collected for these crystals at a temperature of 120K and at room temperature, using a KappaCCD system and MoKα radiation, generated by a FR590 X-ray generator (Bruker Nonius, Delft, The Netherlands).

Unit-cell parameters and crystal structures were determined and refined using the software package MaXus.

### Thermal Analysis:

Melting properties were obtained from DSC thermograms, recorded with a heat flux DSC822e instrument (Mettler-Toledo GmbH, Switzerland). The DSC822e was calibrated for temperature and enthalpy with a small piece of indium (m.p.=156.6° C.; delta-H(f)=28.45 J/g). Samples were sealed in standard 40 microliter aluminum pans and heated in the DSC from 25° C. to 300° C., at a heating rate of 20° C./min. Dry N₂ gas, at a flow rate of 50 ml/min, was used to purge the DSC equipment during measurement.

Mass loss due to solvent or water loss from the crystals was determined by TGA/SDTA. Monitoring of the sample weight, during heating in a TGA/SDTA851e instrument (Mettler-Toledo GmbH, Switzerland), resulted in a weight vs. temperature curve. The TGA/SDTA851e was calibrated for temperature with indium and aluminium. Samples were weighed into 100 microliter aluminium crucibles and sealed. The seals were pin-holed and the crucibles heated in the TGA from 25° C. to 300° C. at a heating rate of 20° C./min. Dry N₂ gas is used for purging. Melting point determinations based on DSC have a variability of +/−2.0 degrees Celsius, preferably 1.0 degrees Celsius.

### Raman Spectroscopy:

The Raman spectra were collected with a Raman microscope mW (Kaiser Opticals Inc) at 0.96 cm⁻¹ resolution using a laser of 780 nm and a power output of 100.

### Examples

The starting material for the crystallisation experiments was obtained as a research sample from Cipla Ltd, Mumbai, India.

Analysis of several commercial samples using the high-resolution X-ray diffractometer:

Commercial samples of Tenofovir were obtained from a local pharmacy (Viread and Truvada) and the coating was carefully removed by scraping or sanding from the surface of the tablet so that the coating material does not contribute to the X-ray diffraction pattern. Two XRPD patterns were collected for Viread with the high resolution X-ray diffractometer from samples differently prepared. The first sample was prepared by a tablet gently ground and the second from a non ground tablet after removal of the coating and flattening of the surface. The XRPD patterns of both samples showed that there was no structural phase transition induced by grinding of the first sample.

The X-ray analysis of Viread indicated that it contains tenofovir DF in Gilead form 1 (as described in U.S. Pat. No. 5,935,946) and the co-crystal of Tenofovir Disoproxil fumarate, TDFA 2:1. All above mentioned XRPD patterns showed also the presence of lactose monohydrate, used as an excipients in both tablets. In Table 3A the 2θ peak positions of the XRPD pattern of the ground tablet of Viread are listed in the first column, together with the peak positions of Gilead form 1 (U.S. Pat. No. 5,935,946) in the second column, the peak positions of the starting material used or the experiments in the third column, the calculated peak positions of TDFA 2:1 (wavelength 1.54056 Å) on the basis of the single crystal structure at room temperature in the fourth column and the calculated peak positions of lactose monohydrate based on the single crystal structure found in the Cambridge Structure Database (REFCODE LACTOS01), in the fifth column.

The same conclusions were drawn when studying the XRPD pattern of Truvada (detailed table not listed here) of which one XRPD pattern of a ground tablet was collected. In that XRPD pattern the 2θ peak positions of emtricitabine were also observed.

TABLE 3A
2θ positions of intensity peaks of the XRPD pattern of the
ground tablet of Viread belonging to Viread Form 1
(U.S. Pat. No. 5,935,946), TDFA 2:1 from single crystal data
and the excipients lactose monohydrate.

| Viread | Gilead Form 1 | | | |
|---|---|---|---|---|
| ground | (U.S. Pat. No. | Starting | Calculated | Lactose |
| tablet | 5,935,946) | material (Cipla) | TDFA 2:1 | monohydrate |
| 4.97 | 4.9 | 4.97 | | |
| 5.44 | | 5.44 | | |
| 7.81 | | | 7.81 | |
| 8.08 | | | 8.08 | 8.19 |
| 9.81 | | | 9.82 | |
| 10.29 | 10.2 | 10.27 | | |
| 10.57 | 10.5 | 10.55 | 10.54 | |
| 10.89 | | 10.88 | 10.95 | |
| 11.42 | | 11.41 | | |
| | | | 11.63 | |
| 11.92 | | | 11.93 | |
| 12.54 | | | 12.58 | 12.50 |
| 14.25 | | | 14.29 | |
| | | | 14.80 | |
| 14.94 | | 14.92 | | |
| 15.34 | | 15.37 | | |
| 16.08 | | 16.14 | 16.07 | |
| 16.34 | | | | |
| 16.44 | | | | 16.43 |
| | | | 16.59 | |
| 16.72 | | | 16.77 | |
| 17.13 | | | 17.20 | 17.07 |
| 17.32 | | 17.32 | | |
| 18.32 | 18.2 | 18.25 | | |
| 19.13 | | | 19.15 | 19.06 |
| 19.55 | | | | 19.50 |
| | | | | 19.86 |
| 19.99 | 20.0 | 19.93 | 19.98 | |
| 20.83 | | | 20.86 | 20.79 |
| 20.93 | 21.9 | | | |
| 21.18 | | | 21.18 | 21.11 |
| 21.25 | | 21.25 | | |
| 22.51 | | | 22.51 | |
| 22.76 | | 22.79 | 22.75 | |
| 23.79 | | | 23.78 | 23.77 |
| | 24.0 | | | |
| 24.24 | | | 24.27 | |
| 24.79 | | | | |
| | 25.0 | 25.00 | | |
| | | | 25.30 | |
| 25.57 | 25.5 | 25.46 | 25.51 | 25.54 |
| | 27.8 | | | |
| 30.07 | 30.1 | 30.11 | 30.03 | |
| | 30.4 | | | |
| 31.05 | | | 31.06 | |
| | | | 31.19 | |
| 34.60 | | | | 34.87 |
| 36.21 | | | | |
| 36.94 | | | | |
| | | | | 37.34 |
| 37.54 | | | 37.50 | |

### Crystallisation of TDFA 2:1 on Microliter Scale.

A small quantity, about 2-3 mg of the commercially available starting material was placed in a plate well. The starting material was stock-dosed in tetrahydrofuran/water (80/20 v/v) mixture. The solvent was removed by evaporation under 20 kPa for about 45-75 h and the starting material was dry. The crystallisation solvent or mixture of crystallisation solvents (50/50 v/v) was added in small amounts to the well containing the dry starting material at room temperature to a total volume of 40 microliter and a stock concentration of 50 or 80 mg/ml. The solution was heated and maintained at 60° C. for 30 minutes. Following, controlled cooling was applied with a cooling rate of about 1° C./h or 50° C./h to a final temperature of 5° C. or 20° C. and remained at this temperature for 1, 48, 75, 117 or 139 h. Subsequently, the solvent was evaporated under pressure of 20 kPa at RT for 48-120 h. The resulting residue was analysed by X-ray powder diffraction, DSC and TG-MS. The solvents employed are in Table I. In a specific experiment in a HPLC vial, 301.6 mg of the starting material was dissolved in 2-methyl-4-pentanol. The solution was heated to 60° C. for 30 minutes. Following, controlled cooling was applied with a cooling rate of about 1° C./h to room temperature (about 22° C.) and remained at this temperature for 48 h. Following, the solid material was separated from the supernatant solution by centrifugation. An XRPD measurements of the solid material showed that it was form D. The supernatant solution was evaporated and XRPD measurement of the residue showed that it was fumaric acid and small amount of form D. This experiments confirms the excess of fumaric acid upon conversion of the 1:1 salt tenofovir DF to the 2:1 co-crystal TDFA 2:1.

### Crystallisation of TDFA 2:1 on Millilitre Scale Using Hot Filtration.

A small quantity, about 70-75 mg of the starting material was placed in a HPLC vial. The crystallisation solvent (or 50/50 v/v mixture of solvents) was added in small amounts to the vial containing the dry starting material at room temperature to a total volume of 200-1000 microliter. The solvents and conditions employed are in Table II. Subsequently, the solutions were heated with a rate of 20 degrees Celsius to 60° C. for 60 min and they were filtered at this temperature. The filtrated solutions were cooled with 1.1 or 50° C./h to a temperature of 3 or 20° C. where they remained for 24 h. Subsequently, the solvents were evaporated from the vial under 20 kPa pressure at 20-25° C. for 15-200 h (see table II, in the case of (R)-(−)-2-Octanol at 0.2 kPa for 500 h). The resulting residue was analysed by X-ray powder diffraction, DSC and TGA.

### Crystallisation of TDFA 2:1 on Millilitre Scale Using Anti-Solvent Addition.

The anti-solvent addition experiments were carried out following two different protocols. According to the first protocol (forward anti-solvent addition) for each solvent, a slurry was prepared at ambient temperature, which was equilibrated for about 17-19 hours before filtering into a vial. The anti-solvent was added, using a solvent:anti-solvent ratio of 1:1. This ratio was increased to 1:4 in those cases where no precipitation occurred, by subsequent anti-solvent additions. The time interval between the additions was 1 h. The total volume of the anti-solvent was equal to that of the saturated solution.

For the second protocol (reverse anti-solvent addition), a slurry was prepared at ambient temperature, which was equilibrated for about 17-19 hours before filtering into a set of four vials. The content of each of these vials was added to a vial containing anti-solvent. The total volume of the four vials of saturated solutions was equal to that of the anti-solvent. The time interval between the additions was 1 h.

Precipitates were recovered by centrifugation, and the solid products were dried and analyzed by XRPD. In the cases that no precipitation occurred the solutions were evaporated for 96-314 hrs at room temperature and the residues were analysed by XRPD.

See Table III for experimental details

### Crystallisation of TDFA 2:1 on Millilitre Scale Using Slurry Crystallisation.

About 50 mg of the starting material was used to make a slurry with a solvent (see Table 4). The slurries were stirred for the time interval of 2 and 10 days at RT or 35° C. as shown in Table 4. The materials were checked by XRPD in order to check for solid form changes. In the XRPD pattern of the material obtained by similar slurry experiments the intensity peaks of fumaric acid were observed at about 28.7°2θ, indicating the excess of fumaric acid in the slurried material as a result of the conversion of the 1:1 tenofovir DF to the 2:1 co-crystal.

TABLE 4
Slurry experiments of Tenofovir DF

| | | Temperature | Time | | Time | |
|---|---|---|---|---|---|---|
| # | Solvent | (° C.) | (days) | Form | (days) | form |
| 1 | water | RT | 2 | TDFA 2:1 | 10 | TDFA 2:1 |
| 2 | water | 35 | 2 | TDFA 2:1 | 10 | TDFA 2:1 |

Slurry experiments in water both at room temperature and at 35° C. led to the conversion of the starting material to form TDFA 2:1 after 2 days. An XRPD measurement of the materials in slurries after 10 days showed that the solid form was still form TDFA 2:1.

Slurry experiments of the starting material in 1,4-dioxane and acetonitrile at RT did not lead to any solid form conversion after 2 and 10 days.

### Crystallisation of TDFA 2:1 on Millilitre Scale Using Seeding Crystallisation.

Three types of seeding experiments were performed as described below:

### Type 1

A slurry was made at RT using about 100 mg of the starting material. The slurry was filtered at RT and a small quantity of about 2 mg of the corresponding seed was added. The solution remained at RT or 5° C. overnight. Subsequently the solution was evaporated and the solid material was checked by XRPD.

### Type 2

The experiments were performed as described in the anti-solvent addition example with the following modification: immediately after precipitation, a small quantity of about 2 mg of the corresponding seed was added to the solution. The solution remained at RT or 5° C. overnight. Subsequently the solution was evaporated and the solid material was checked by XRPD.

### Type 3

A slurry was made at RT using about 100 mg of the starting material. A small quantity of about 5 mg of the corresponding seed was added. The slurry was stirred for about 1 h and there after it remained at RT for 2 days. Subsequently the solution was evaporated and the solid material was checked by XRPD.

The specific conditions and seeds used in each experiment are listed in Table 5

TABLE 5
Seeding experiments performed using commercially available
Tenofovir DF. In all solvent mixtures the ration was 50/50. The anti-
solvent addition was reverse as described in the corresponding
paragraph.

| Exp | solvent | anti solvent | type | Seed | Result |
|---|---|---|---|---|---|
| 15 | 2-ethoxyethylacetate | | 1 | TDFA 2:1 | TDFA 2:1 |
| 16 | 2-ethoxyethylacetate | | 1 | TDFA 2:1 | TDFA 2:1 |
| 18 | 2-methyl-4-pentanol | | 1 | TDFA 2:1 | TDFA 2:1 |
| 19 | water | | 3 | TDFA 2:1 | TDFA 2:1 |
| 20 | tetrahydrofuran | amylether | 2 | TDFA 2:1 | TDFA 2:1 |
| 22 | tetrahydrofuran | tert-butyl | 2 | TDFA 2:1 | TDFA 2:1 |
| | | methyl ether | | | |
| 23 | tetrahydrofuran | tert-butyl | 2 | TDFA 2:1 | TDFA 2:1 |
| | | methyl ether | | | |

### Crystallization of TDFA 2:1 on Milliliter Scale.

From 2,2,2 trifluoroethanol:

A small quantity, about 15.8 mg of the starting material was placed in a HPLC vial. The solvent 2,2,2-trifluoroethanol was added in small amounts to the vial containing the dry starting material at room temperature to a total volume of 1000 microliter. The vial was shaken and the qualitative solubility was assessed visually. The solution was heated and maintained at 60° C. for 30 minutes. Subsequently, the solvent was evaporated from the vial under vacuum at 20-25° C. The evaporation time and pressure was 22.5 hr at 20 KPa. Evaporation was continued for 71 hr at 4.4 KPa. The resulting residue was analyzed by X-ray powder diffraction, DSC and TGA and identified as TDFA 2:1

### From Acetone:

A small quantity, about 15.3 mg of the starting material was placed in a HPLC vial. The solvent acetone was added in small amounts to the vial containing the dry starting material at room temperature to a total volume of 1000 microliter. The vial was shaken and the qualitative solubility was assessed visually. Subsequently, the solvent was evaporated from the vial under vacuum at 20-25° C. The evaporation time and pressure was 22.5 hr at 20 KPa. The resulting residue was analyzed by X-ray powder diffraction, DSC and TGA and identified as TDFA 2:1

### From Dichloromethane:

A small quantity, about 12.4 mg of the starting material was placed in a HPLC vial. The solvent dichloromethane was added in small amounts to the vial containing the dry starting material at room temperature to a total volume of 1000 microliter. The vial was shaken and the qualitative solubility was assessed visually. The solution was heated and maintained at 60° C. for 30 minutes. Subsequently, the solvent was evaporated from the vial under vacuum at 20-25° C. The evaporation time and pressure was 22.5 hr at 20 KPa. The resulting residue was analyzed by X-ray powder diffraction, DSC and TGA and identified as Tenofovir DF form TDFA 2:1

### From Nitromethane:

A small quantity, about 15.9 mg of the starting material was placed in a HPLC vial. The solvent nitromethane was added in small amounts to the vial containing the dry starting material at room temperature to a total volume of 1000 microliter. The vial was shaken and the qualitative solubility was assessed visually. The solution was heated and maintained at 60° C. for 30 minutes. Subsequently, the solvent was evaporated from the vial under vacuum at 20-25° C. The evaporation time and pressure was 22.5 hr at 20 KPa. The resulting residue was analyzed by X-ray powder diffraction, DSC and TGA and identified as Tenofovir DF form TDFA 2:1

### From Water:

A small quantity, about 16.9 mg of the starting material was placed in a HPLC vial. The solvent water was added in small amounts to the vial containing the dry starting material at room temperature to a total volume of 1000 microliter. The vial was shaken and the qualitative solubility was assessed visually. The solution was heated and maintained at 60° C. for 30 minutes. Subsequently, the solvent was evaporated from the vial under vacuum at 20-25° C. The evaporation time and pressure was 22.5 hr at 20 KPa. Evaporation was continued for 71 hr at 4.4 KPa. The resulting residue was analyzed by X-ray powder diffraction, DSC and TGA and identified as Tenofovir DF form TDFA 2:1.

### Dynamic Vapour Sorption (DVS)

Moisture sorption isotherms were measured using a DVS-1 system of Surface Measurement Systems (London, UK). Differences in moisture uptake of various forms of a solid material indicate differences in the relative stabilities of the various solid forms for increasing relative humidity. The experiment was carried out at a constant temperature of 25° C.

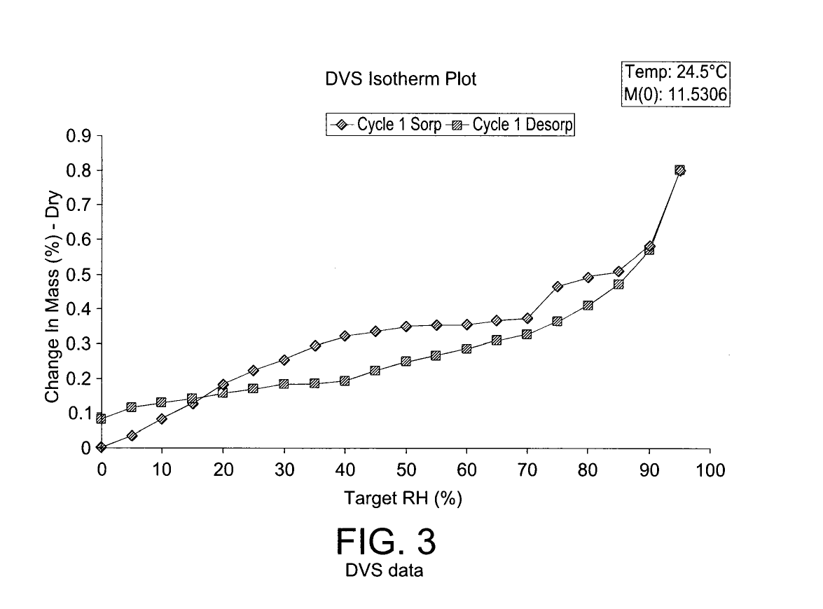

A sample of about 11.5 mg of form TDFA 2:1 was spread in the DVS pan. The sample was dried at 0% RH for 7 h. Subsequently the relative humidity of the chamber was increased in steps of 5% units from 0% to 95% in order to monitor the sorption of water vapours. The samples remained in each of the steps for 1 h. Following, desorption was monitored by decreasing the relative humidity to 0% in steps of 5% units and remaining at each step for 1 h. The graph of sorption-desorption cycle is shown in FIG. 3. The total uptake of water vapours was about 0.8%, which is line with the industry standard for hygroscopicity. In a similar DVS experiment using the starting material as purchased, the total vapour intake was about 4%, which is undesirable in formulation and requires additional measures.

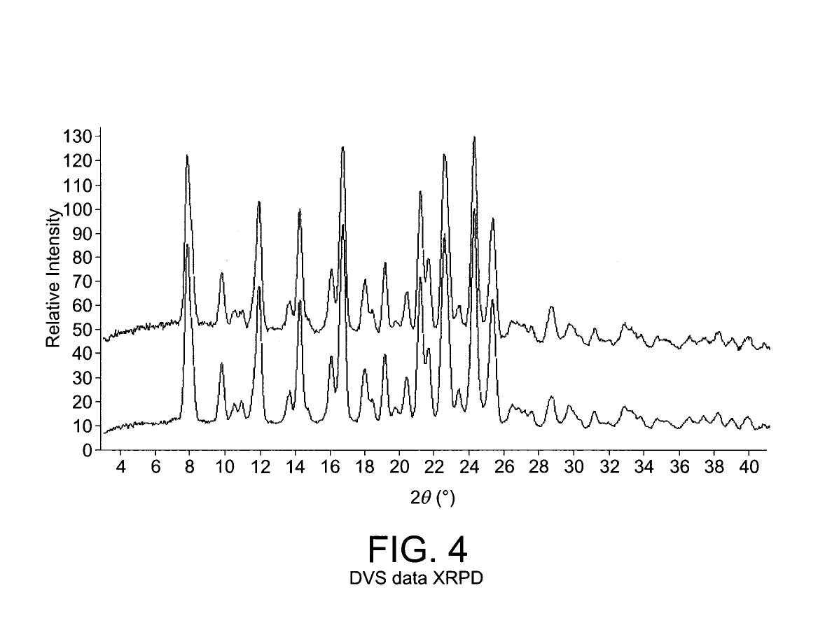

At the end of the experiment, the solid material was measured by XRPD which showed that there were no any changes in the structure (FIG. 4).

### Example

### Comparative Pharmacokinetic Study of TDFA 2:1 and ULT 1

Batches of TDFA 2:1 and ULT 1 were prepared with comparable crystal size by sieving through a μM sieve. Small cellulose capsules were filled with approximately 15 mg of either tenofovir DF form TDFA 2:1 or tenofovir ULT Y. Twelve Male wistar rats of approximately 300 grams each were dosed one capsule with either form TDFA 2:1 or ULT 1 by oral gavage followed by 1 mL of tap water. At regular intervals a small quantity of blood was sampled from each rat by a tail vein puncture. Blood samples were immediately frozen in Liquid N2 for further processing.

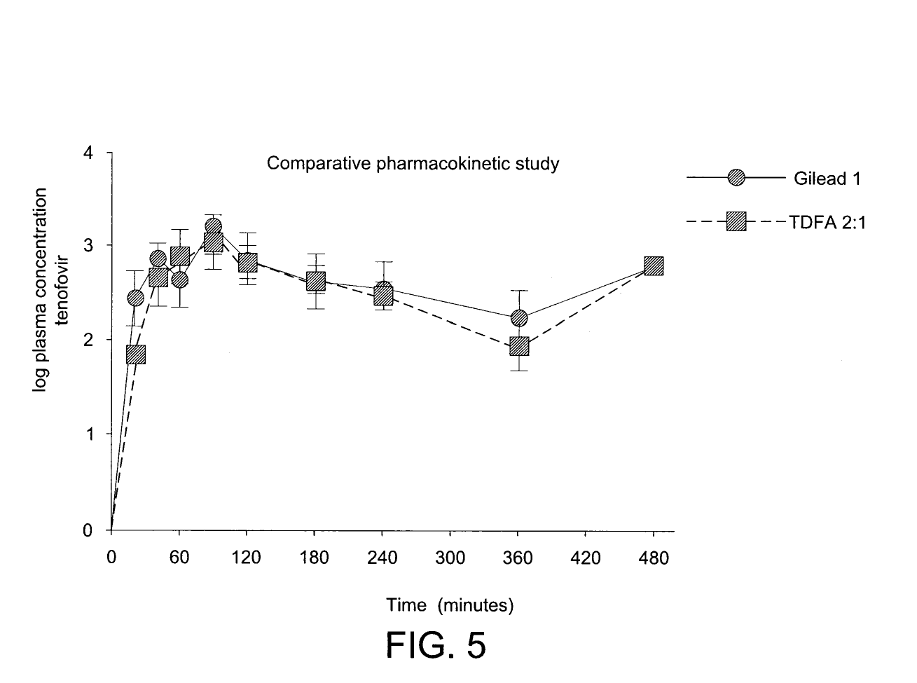

After all samples have been collected, plasma preparations were made of each sample. The plasma samples were further worked up for analysis by LC-MS-MS for their content of tenofovir. Efficiency of extraction was determined by comparison by spiking rat plasma samples with known amounts of tenofovir. The concentration of tenofovir (the active metabolite of tenofovir DF) was quantified in each sample by means of LC-MS-MS against a calibration curve. The results of the comparative pharmacokinetic are presented in FIG. 5. From the PK data it is concluded that the hemifumarate co-crystal of tenofovir disoproxil, TDFA 2:1 is bioequivalent to the fumarate salt of tenofovir disoproxil form ULT 1, which is a commercially available fumarate salt of tenofovir disoproxil.

TABLE 3
Final Co-ordinates and Equivalent Isotropic Displacement of
tenofovir DF form TDFA 2:1

| Atom | x | y | z | U(eq) [Ang²] |
|---|---|---|---|---|
| P16B | 0.91345(3) | 0.26263(1) | 1.20742(2) | 0.0156(1) |
| O14B | 1.09447(8) | 0.23927(3) | 1.06423(6) | 0.0169(2) |
| O17B | 0.89354(9) | 0.29986(4) | 1.30247(7) | 0.0223(2) |
| O18B | 0.88847(9) | 0.19217(4) | 1.22344(7) | 0.0202(2) |
| O20B | 0.79833(10) | 0.16996(4) | 1.38678(8) | 0.0277(2) |
| O22B | 0.68433(12) | 0.09767(6) | 1.28859(10) | 0.0422(3) |
| O23B | 0.60394(13) | 0.13936(6) | 1.43391(10) | 0.0454(4) |
| O27B | 0.81499(8) | 0.27486(4) | 1.10195(7) | 0.0188(2) |
| O29B | 0.59441(8) | 0.29274(4) | 1.15326(7) | 0.0216(2) |
| O31B | 0.61646(9) | 0.37047(5) | 1.03740(7) | 0.0265(2) |
| O32B | 0.50211(11) | 0.37824(4) | 1.18655(8) | 0.0298(3) |
| O41 | 0.01933(8) | 0.50567(4) | 0.65585(8) | 0.0233(2) |
| O43 | −0.15664(9) | 0.45436(5) | 0.57261(9) | 0.0323(3) |
| O47 | −0.34693(9) | 0.54302(5) | 0.90416(9) | 0.0304(3) |
| O48 | −0.51806(8) | 0.48512(4) | 0.83015(7) | 0.0208(2) |
| N1B | 0.52743(9) | 0.09355(4) | 0.95360(8) | 0.0196(2) |
| N3B | 0.69018(9) | 0.04230(4) | 1.06597(8) | 0.0167(2) |
| N5B | 0.93231(9) | 0.06195(4) | 1.08033(8) | 0.0175(2) |
| N7B | 0.98861(8) | 0.14178(4) | 0.95782(7) | 0.0145(2) |
| N9B | 0.77594(9) | 0.16564(4) | 0.88479(8) | 0.0173(2) |
| C2B | 0.65806(10) | 0.08551(4) | 0.99080(8) | 0.0146(2) |
| C4B | 0.82168(11) | 0.03351(5) | 1.10732(9) | 0.0183(3) |
| C6B | 0.90024(9) | 0.10404(4) | 1.00403(8) | 0.0136(2) |
| C8B | 0.90826(10) | 0.17780(5) | 0.88869(9) | 0.0170(2) |
| C10B | 0.77001(10) | 0.11860(4) | 0.95721(8) | 0.0142(2) |
| C11B | 1.13734(10) | 0.14644(5) | 0.98087(8) | 0.0162(2) |
| C12B | 1.17537(10) | 0.18546(5) | 1.07956(8) | 0.0152(2) |
| C13B | 1.32780(11) | 0.20033(6) | 1.08974(10) | 0.0218(3) |
| C15B | 1.08074(10) | 0.27277(5) | 1.15925(9) | 0.0178(2) |
| C19B | 0.91274(12) | 0.16332(6) | 1.32523(10) | 0.0235(3) |
| C21B | 0.69179(13) | 0.13198(5) | 1.36107(10) | 0.0226(3) |
| C24B | 0.47573(15) | 0.10351(7) | 1.42046(13) | 0.0336(4) |
| C25B | 0.3600(2) | 0.14540(8) | 1.38284(15) | 0.0412(5) |
| C26B | 0.45894(19) | 0.07468(8) | 1.52771(17) | 0.0418(5) |
| C28B | 0.67480(11) | 0.25688(5) | 1.08862(10) | 0.0219(3) |
| C30B | 0.57460(11) | 0.35039(5) | 1.11745(9) | 0.0210(3) |
| C33B | 0.48079(16) | 0.44348(6) | 1.16884(13) | 0.0327(4) |
| C34B | 0.5967(3) | 0.47536(10) | 1.2292(3) | 0.0704(11) |
| C35B | 0.3415(2) | 0.45688(9) | 1.20439(17) | 0.0464(5) |
| C42 | −0.10761(11) | 0.48694(5) | 0.64591(10) | 0.0200(3) |
| C44 | −0.18877(10) | 0.50845(5) | 0.73399(9) | 0.0187(3) |
| C45 | −0.31537(11) | 0.48798(5) | 0.74395(9) | 0.0184(3) |
| C46 | −0.39556(11) | 0.50771(5) | 0.83428(9) | 0.0187(3) |
| P16A | 0.42931(3) | 0.23205(1) | 0.74184(2) | 0.0157(1) |
| O14A | 0.60472(7) | 0.29201(3) | 0.63165(6) | 0.0161(2) |
| O17A | 0.40418(9) | 0.17319(4) | 0.79058(8) | 0.0234(2) |
| O18A | 0.32787(8) | 0.25070(4) | 0.64071(7) | 0.0201(2) |
| O20A | 0.12135(8) | 0.21228(4) | 0.69565(7) | 0.0201(2) |
| O22A | 0.11388(12) | 0.15029(5) | 0.54987(8) | 0.0320(3) |
| O23A | 0.05859(9) | 0.12016(4) | 0.71428(7) | 0.0242(2) |
| O27A | 0.42651(9) | 0.28315(4) | 0.83038(7) | 0.0212(2) |
| O29A | 0.28255(8) | 0.36500(4) | 0.79005(7) | 0.0218(2) |
| O31A | 0.25326(10) | 0.35807(5) | 0.96887(8) | 0.0287(3) |
| O32A | 0.07710(9) | 0.37019(4) | 0.83825(7) | 0.0239(2) |
| N1A | 0.02660(9) | 0.39113(5) | 0.44272(8) | 0.0197(2) |
| N3A | 0.18775(9) | 0.44258(4) | 0.55637(7) | 0.0160(2) |
| N5A | 0.43128(9) | 0.42542(4) | 0.56736(8) | 0.0168(2) |
| N7A | 0.48955(8) | 0.34626(4) | 0.44440(7) | 0.0146(2) |
| N9A | 0.27694(9) | 0.31945(4) | 0.37477(8) | 0.0181(2) |
| C2A | 0.15734(10) | 0.39929(4) | 0.48082(8) | 0.0145(2) |
| C4A | 0.31933(11) | 0.45261(5) | 0.59563(9) | 0.0175(2) |
| C6A | 0.39987(10) | 0.38259(4) | 0.49233(8) | 0.0136(2) |
| C8A | 0.40997(11) | 0.30952(5) | 0.37572(9) | 0.0173(2) |
| C10A | 0.26918(10) | 0.36627(4) | 0.44756(8) | 0.0141(2) |
| C11A | 0.63828(10) | 0.34312(5) | 0.46991(8) | 0.0154(2) |
| C12A | 0.67760(10) | 0.28760(5) | 0.53708(8) | 0.0148(2) |
| C13A | 0.83136(11) | 0.28307(6) | 0.56511(9) | 0.0205(3) |
| C15A | 0.59062(10) | 0.23559(5) | 0.68317(9) | 0.0190(3) |
| C19A | 0.18263(10) | 0.25815(6) | 0.63666(10) | 0.0215(3) |
| C21A | 0.09977(11) | 0.15889(5) | 0.64315(9) | 0.0213(3) |
| C24A | 0.00839(16) | 0.06125(6) | 0.67243(12) | 0.0315(3) |
| C25A | 0.0193(2) | 0.01971(7) | 0.76906(15) | 0.0392(5) |
| C26A | −0.1373(2) | 0.06938(9) | 0.62337(16) | 0.0453(5) |
| C28A | 0.42197(12) | 0.34723(5) | 0.81145(10) | 0.0210(3) |
| C30A | 0.20729(12) | 0.36409(5) | 0.87681(9) | 0.0215(3) |
| C33A | −0.02724(12) | 0.36042(5) | 0.91567(9) | 0.0213(3) |
| C34A | −0.04266(14) | 0.41619(6) | 0.98370(12) | 0.0278(3) |
| C35A | −0.15684(13) | 0.34406(6) | 0.84660(10) | 0.0248(3) |
| H1B1 | 0.4641(18) | 0.0755(8) | 0.9823(15) | 0.022(4) |
| H1B2 | 0.5069(19) | 0.1243(9) | 0.9050(15) | 0.027(4) |
| H4B | 0.8276(16) | 0.0034(8) | 1.1602(13) | 0.017(4) |
| H8B | 0.9501(16) | 0.2076(7) | 0.8464(13) | 0.016(3) |
| H11C | 1.1712(17) | 0.1634(8) | 0.9183(14) | 0.020(4) |
| H11D | 1.1763(16) | 0.1064(7) | 0.9877(13) | 0.018(4) |
| H12B | 1.1444(17) | 0.1644(7) | 1.1439(13) | 0.018(4) |
| H13D | 1.3485(16) | 0.2291(8) | 1.1481(13) | 0.018(4) |
| H13E | 1.3537(18) | 0.2207(8) | 1.0225(15) | 0.028(4) |
| H13F | 1.386(2) | 0.1648(9) | 1.1048(16) | 0.035(5) |
| H15C | 1.0911(18) | 0.3142(8) | 1.1423(14) | 0.022(4) |
| H15D | 1.1469(18) | 0.2654(9) | 1.2206(15) | 0.027(4) |
| H19C | 0.9328(19) | 0.1209(9) | 1.3130(15) | 0.028(4) |
| H19D | 0.9881(18) | 0.1821(8) | 1.3680(14) | 0.026(4) |
| H24B | 0.4878(18) | 0.0746(9) | 1.3668(15) | 0.028(4) |
| H25D | 0.386(3) | 0.1593(12) | 1.313(2) | 0.063(7) |
| H25E | 0.349(2) | 0.1804(9) | 1.4329(16) | 0.034(5) |
| H25F | 0.271(2) | 0.1226(10) | 1.3684(17) | 0.043(6) |
| H26D | 0.547(2) | 0.0497(11) | 1.5536(19) | 0.051(6) |
| H26E | 0.376(3) | 0.0467(12) | 1.518(2) | 0.057(7) |
| H26F | 0.438(2) | 0.1058(11) | 1.5813(18) | 0.046(6) |
| H28C | 0.6459(16) | 0.2612(7) | 1.0150(13) | 0.017(3) |
| H28D | 0.6639(16) | 0.2174(7) | 1.1131(13) | 0.016(4) |
| H33B | 0.483(2) | 0.4513(11) | 1.0882(18) | 0.045(6) |
| H34D | 0.677(5) | 0.461(2) | 1.211(4) | 0.139(16) |
| H34E | 0.599(3) | 0.4694(16) | 1.315(3) | 0.089(11) |
| H34F | 0.587(3) | 0.5152(13) | 1.224(2) | 0.061(7) |
| H35D | 0.268(3) | 0.4302(13) | 1.162(2) | 0.072(9) |
| H35E | 0.324(3) | 0.4986(12) | 1.192(2) | 0.055(7) |
| H35F | 0.328(3) | 0.4434(15) | 1.273(3) | 0.083(9) |
| H41 | 0.075(4) | 0.4783(18) | 0.605(3) | 0.115(13) |
| H44 | −0.1451(18) | 0.5351(8) | 0.7865(14) | 0.022(4) |
| H45 | −0.3605(18) | 0.4609(9) | 0.6966(14) | 0.027(4) |
| H48 | −0.601(4) | 0.5100(18) | 0.889(3) | 0.115(13) |
| H1A1 | 0.0023(19) | 0.3624(9) | 0.3965(15) | 0.026(4) |
| H1A2 | −0.034(2) | 0.4105(9) | 0.4752(15) | 0.030(5) |
| H4A | 0.3340(18) | 0.4842(8) | 0.6493(15) | 0.025(4) |
| H8A | 0.4547(16) | 0.2809(7) | 0.3324(13) | 0.017(4) |
| H11A | 0.6680(16) | 0.3796(7) | 0.5100(13) | 0.016(3) |
| H11B | 0.6830(18) | 0.3419(8) | 0.4040(14) | 0.023(4) |
| H12A | 0.6478(15) | 0.2518(7) | 0.4977(13) | 0.014(3) |
| H13A | 0.8497(19) | 0.2490(9) | 0.6082(16) | 0.031(5) |
| H13B | 0.8731(18) | 0.3240(8) | 0.6003(14) | 0.023(4) |
| H13C | 0.8816(18) | 0.2808(8) | 0.4959(14) | 0.024(4) |
| H15A | 0.663(2) | 0.2275(9) | 0.7384(15) | 0.032(5) |
| H15B | 0.5875(18) | 0.1987(8) | 0.6300(15) | 0.025(4) |
| H19A | 0.1604(17) | 0.2968(8) | 0.6716(14) | 0.021(4) |
| H19B | 0.1502(18) | 0.2568(8) | 0.5650(15) | 0.024(4) |
| H24A | 0.063(2) | 0.0497(9) | 0.6183(16) | 0.033(5) |
| H25A | 0.111(2) | 0.0179(10) | 0.7964(18) | 0.043(6) |
| H25B | −0.010(3) | −0.0197(12) | 0.748(2) | 0.056(7) |
| H25C | −0.045(2) | 0.0310(11) | 0.825(2) | 0.052(6) |
| H26A | −0.140(3) | 0.0948(14) | 0.563(2) | 0.074(8) |
| H26B | −0.192(2) | 0.0866(11) | 0.684(2) | 0.053(7) |
| H26C | −0.168(3) | 0.0319(12) | 0.598(2) | 0.055(7) |
| H28A | 0.4648(17) | 0.3634(8) | 0.8756(14) | 0.021(4) |
| H28B | 0.4682(17) | 0.3583(8) | 0.7496(14) | 0.022(4) |
| H33A | 0.0111(18) | 0.3248(8) | 0.9610(14) | 0.023(4) |
| H34A | 0.038(2) | 0.4261(10) | 1.0248(17) | 0.038(5) |
| H34B | −0.114(2) | 0.4100(11) | 1.0322(18) | 0.047(6) |
| H34C | −0.0691(19) | 0.4508(10) | 0.9407(16) | 0.033(5) |
| H35A | −0.1430(19) | 0.3064(9) | 0.8055(15) | 0.030(4) |
| H35B | −0.187(2) | 0.3776(10) | 0.7938(17) | 0.042(5) |
| H35C | −0.234(2) | 0.3358(9) | 0.8943(16) | 0.033(5) |

TABLE I

| Form | | Solvent 1 | Solvent 2 | % solvent 1 |
|---|---|---|---|---|
| TDFA 2:1 | D | N-Methyl Pyrrolidone | Tetrahydrofuran | 50 |
| TDFA 2:1 | D | N-Methyl Pyrrolidone | Water | 50 |
| TDFA 2:1 | D | 1,4-Dioxane | N-Methyl | 50 |
| | | | Pyrrolidone | |
| TDFA 2:1 | D | 1-Heptanol | — | — |
| TDFA 2:1 | D | Nitrobenzene | Tetrahydrofuran | 50 |
| TDFA 2:1 | D | 1,2-Dimethoxyethane | N-Methyl | 50 |
| | | | Pyrrolidone | |
| TDFA 2:1 | D | Nitrobenzene | Tetrahydrofuran | 50 |
| TDFA 2:1 | D | 1-Hexanol | — | — |
| TDFA 2:1 | D | 2-Pentanol | — | — |
| TDFA 2:1 | D | 1-Heptanol | — | — |
| TDFA 2:1 | D | 1-Octanol | Methanol | 50 |
| TDFA 2:1 | D | 1-Hexanol | — | — |
| TDFA 2:1 | D | 1-Hexanol | — | — |
| TDFA 2:1 | D | N-Methyl Pyrrolidone | Water | 50 |

TABLE II

| | | | | (mg/mL) | Evap- |
|---|---|---|---|---|---|
| | | | | Stock | oration |
| | | | | Con- | Time |
| Form | | solvent 1 | Solvent 2 | centration | (hrs) |
| TDFA 2:1 | D | 2-Ethoxyethyl acetate | — | 119 | 70.5 |
| TDFA 2:1 | D | 2-Methyl-4-pentanol | — | 89.6 | 70.5 |
| TDFA 2:1 | D | 2-Ethoxyethyl acetate | — | 119 | 70.5 |
| TDFA 2:1 | D | 2-Propanol | — | 238.3 | 15.1 |
| TDFA 2:1 | D | 2-Ethoxyethyl acetate | — | 119 | 21.5 |
| TDFA 2:1 | D | 2-Ethoxyethyl acetate | — | 119 | 21.5 |
| TDFA 2:1 | D | (R)-(−)-2-Octanol | — | 73.7 | 21.5 |
| TDFA 2:1 | D | 2-Methyl-4-pentanol | — | 89.6 | 70.5 |
| TDFA 2:1 | D | 1-Butanol | — | 161.1 | 15.1 |

TABLE III

| | | | | | Anti |
|---|---|---|---|---|---|
| | | | | | solvent- |
| | | | | | Solvent |
| | | | (mg/mL) Stock | | Ratio |
| Form | | Solvent 1 | Concentration | Anti solvent | (x:1) |
| TDFA 2:1 | D | 2-Butanone | 36.4 | Amyl ether | 1 |
| TDFA 2:1 | D | Tetrahydrofuran | 66.8 | 2,6-Dimethyl-4-heptanone | 1 |
| TDFA 2:1 | D | 1,2-Dimethoxyethane | 36.3 | Amyl ether | 1 |
| TDFA 2:1 | D | 1,2-Dimethoxyethane | 36.3 | Amyl ether | 1 |
| TDFA 2:1 | D | Tetrahydrofuran | 66.8 | tert-Butyl methyl ether | 2 |
| TDFA 2:1 | D | Tetrahydrofuran | 66.8 | tert-Butyl methyl ether | 1 |
| TDFA 2:1 | D | Tetrahydrofuran | 66.8 | Amyl ether | 1 |
| TDFA 2:1 | D | 1,2-Dimethoxyethane | 36.3 | 2,6-Dimethyl-4-heptanone | 1 |
| TDFA 2:1 | D | Tetrahydrofuran | 66.8 | 2,6-Dimethyl-4-heptanone | 1 |
| TDFA 2:1 | D | 1,2-Dimethoxyethane | 36.3 | tert-Butyl methyl ether | 4 |
| TDFA 2:1 | D | Tetrahydrofuran | 66.8 | Amyl ether | 1 |
| TDFA 2:1 | D | N-Methyl Pyrrolidone | 504.6 | tert-Butyl methyl ether | 3 |
| TDFA 2:1 | D | N-Methyl Pyrrolidone | 504.6 | tert-Butyl methyl ether | 1 |
| TDFA 2:1 | D | 1,2-Dimethoxyethane | 36.3 | Cyclohexane | 1 |
| TDFA 2:1 | D | 1,2-Dimethoxyethane | 36.3 | tert-Butyl methyl ether | 3 |
| TDFA 2:1 | D | 2-Butanone | 36.4 | Cyclohexane | 1 |
| TDFA 2:1 | D | N-Methyl Pyrrolidone | 504.6 | 1,2-Dichloroethane | 2 |
| TDFA 2:1 | D | 1,2-Dimethoxyethane | 36.3 | 2,6-Dimethyl-4-heptanone | 1 |
| TDFA 2:1 | D | 2-Butanone | 36.4 | 2,6-Dimethyl-4-heptanone | 2 |
| TDFA 2:1 | D | 1,2-Dimethoxyethane | 36.3 | n-Heptane | 1 |
| TDFA 2:1 | D | N-Methyl Pyrrolidone | 504.6 | Hexafluorobenzene | 1 |
| TDFA 2:1 | D | 2-Butanone | 36.4 | 2,6-Dimethyl-4-heptanone | 2 |
| TDFA 2:1 | D | N-Methyl Pyrrolidone | 504.6 | 1,2-Dichloroethane | 4 |

1. A composition of tenofovir disoproxil with fumaric acid wherein the ratio of tenofovir disoproxil to fumaric acid is about 2:1 (TDFA 2:1).

2. composition according to claim 1, which is a co-crystal.

3. Co-crystal TDFA 2:1 according to claim 1, wherein the co-crystal is a co-crystal at temperatures between at 120 K and room temperature.

4. Co-crystal TDFA 2:1 according to claim 2, characterised by one or more of:
a XRPD pattern substantially as set out in Table 1 and/or FIG. 1A;
a DSC substantially as set out in FIG. 1B;
a TGA substantially as set out in FIG. 1C;
a single crystal structure substantially as set out in FIG. 1E.

5. Co-crystal TDFA 2:1 according to claim 2 in a substantially pure form.

6. Method for the preparation of co-crystal TDFA 2:1, comprising the steps of
dissolving or mixing tenofovir DF in a suitable solvent or mixture thereof as in Table I and crystallising Co-crystal TDFA 2:1 by evaporation of the solvent; and/or
dissolving or mixing tenofovir DF in a suitable solvent or mixture thereof as in Table II and crystallising Co-crystal TDFA 2:1 by cooling and/or evaporation crystallization of a saturated solution; and/or
dissolving or mixing tenofovir DF in a suitable solvent or mixture thereof as in Table III and crystallising Co-crystal TDFA 2:1 by anti-solvent addition as in Table III; and/or
dissolving or mixing tenofovir DF in a suitable solvent or mixture thereof and crystallising tenofovir DF Form TDFA 2:1 by slurry crystallisation and/or seed crystallisation.

7. Co-crystal TDFA 2:1, characterized by one or more of:
at least one, preferably at least two, more preferably at least three, even more preferably at least four X-ray powder diffraction peaks selected from the group consisting of 7.9, 9.8, 11.0, 12.0, 13.7, 14.3, 16.1, 16.8, 18.0, 19.2, 20.4, 21.2, 21.7, 22.6, 23.4, 24.3, 25.4, 27.6, degrees two-theta+/−0.3 degrees two-theta, preferable about 0.2 degrees, more preferably 0.1 degrees, even more preferable 0.05 degrees;
DSC with a characterizing peak at 117.0+/−2° C.

8. Method for the preparation of the Co-crystal TDFA 2:1 comprising the steps of dissolving Tenofovir DF in 2,2,2-trifluoroethanol, acetone, dichloromethane, nitromethane or water and crystallizing Co-crystal TDFA 2:1 by evaporation of the solvent.

9. Method for the preparation of the co-crystal TDFA 2:1, comprising the steps of
dissolving or mixing tenofovir disoproxil fumarate 1:1 in a suitable solvent or mixture thereof as in Table I and crystallising Co-crystal TDFA 2:1 by evaporation of the solvent; and/or
dissolving or mixing tenofovir disoproxil fumarate 1:1 in a suitable solvent or mixture thereof as in Table II and crystallising Co-crystal TDFA 2:1 by cooling and/or evaporation crystallization of a saturated solution; and/or
dissolving or mixing tenofovir disoproxil fumarate 1:1 in a suitable solvent or mixture thereof as in Table III and crystallising Co-crystal TDFA 2:1 by anti-solvent addition as in Table III; and/or
dissolving or mixing tenofovir disoproxil fumarate 1:1 in a suitable solvent or mixture thereof and crystallising tenofovir DF Form TDFA 2:1 by slurry crystallisation and/or seed crystallisation.

10. Method according to claim 9 wherein the solvent is an aqueous solvent, preferably water.

11. Method for the preparation of co-crystal TDFA 2:1 in a substantially pure form, comprising contacting tenofovir disoproxil fumarate 1:1 with an aqueous solvent, preferably water.

12. Pharmaceutical formulation comprising TDFA 2:1, which is substantially free from a solid form characterised by having an X-ray peak at 5.5 degrees two-theta+/−0.3 degrees two-theta and preferably essentially in absence of form Gilead 1.

13. Use of Co-crystal TDFA 2:1 essentially in absence of form Gilead 1 as a medicament.

14. Use of Co-crystal TDFA 2:1 essentially in absence of form Gilead 1 in the preparation of a medicament for the treatment of HIV.

15. Use of Co-crystal TDFA 2:1 essentially in absence of form Gilead 1 in the treatment of HIV.

16. Use of Co-crystal TDFA 2:1 essentially in absence of form Gilead 1 in combination with another pharmaceutical ingredient, preferably an anti HIV agent, preferably Efavirenz and/or Emtricitabine and/or TMC114.
